(19) United States
(12) Patent Application Publication
(10) Pub. No.: US 2011/0071626 A1
(43) Pub. Date: Mar. 24, 2011

(54) Device and Method for the Surgical Treatment of Ischemic Mitral Regurgitation

(76) Inventors: John T.M. Wright, Denver, CO (US); Thoralf M. Sundt, Rochester, MN (US); Maurice Enriquez-Sarano, Rochester, MN (US)

(21) Appl. No.: 12/992,293

(22) PCT Filed: May 12, 2009
(86) PCT No.: PCT/US09/43660

§ 371 Date: Nov. 12, 2010

Related U.S. Application Data
(60) Provisional application No. 61/052,590, filed on May 12, 2008.
(60) Provisional application No. 61/096,521, filed on Sep. 12, 2008.
(60) Provisional application No. 61/060,712, filed on Jun. 11, 2008.

(51) Int. Cl. A61F 2/24

(52) U.S. Cl. 623/2.37

(57) ABSTRACT

A device for the treatment of ischemic mitral regurgitation of a damaged left ventricle having an opposing papillary muscles displaced laterally or axially relative to the mitral annulus. The device includes a lateral limb configured to extend between opposing papillary muscles of a left ventricle and first and second axial limbs extending from spaced points on the lateral limb to form an essentially U-shaped configuration. The first and second axial limbs are configured to extend between the lateral limb and a mitral annulus. A method for the treatment of mitral regurgitation comprises extending a first segment of the lateral limb of the device across opposing sides of a ventricle near opposing papillary muscles. The segment of the first lateral limb is shortened as needed to put the first and second papillary muscles into a substantially normal lateral position relative to the mitral annulus and the length of the segment of the first lateral limbs is fixed. The method further includes extending a segment of the first axial limb between the lateral limb and the mitral annulus and extending a segment of the second axial limb between the lateral limb and the mitral annulus. The first and second axial limb segments are then shortened, as needed, to put opposing papillary muscles into a substantially normal axial position relative to the mitral annulus and the lengths of the segments of the first and second axial limbs are fixed.

### TECHNICAL FIELD

The present invention relates generally to surgical treatment of heart disease, and more specifically to a device and method for the surgical treatment of ischemic (functional) mitral regurgitation.

### BACKGROUND

Ischemic mitral regurgitation (IMR), also called functional mitral regurgitation, is caused by a damaged left ventricular wall following myocardial infarct. Following the infarct the left ventricular wall in the area of the infarct becomes thinned, bulges outwardly and is either akinetic or has reduced contractility. Hence the left ventricle becomes enlarged and, the adjacent papillary muscle or muscles move outwardly. Also, because the ventricular wall is stretched, the papillary muscle often moves axially towards the left ventricular apex relative to the mitral annulus so that the distances between the papillary muscle(s) and the mitral annulus increase markedly. In turn, the papillary muscles displace the bases of the chordae tendineae. The chordae connect the mitral valve leaflets to the papillary muscles. Therefore the coaptive area of the mitral leaflets is pulled downwards and outwards so that the area of coaptation of the anterior to posterior leaflets becomes first reduced and later lost. When coaptation is lost the valve leaflets fail to meet during systole and the valve becomes regurgitant. The pulling of the leaflets downwards and outwards is called “tenting”. In advance IMR the ventricle become somewhat spherical in shape rather than its normal ellipsoidel form. The incidence of IMR in the United States is estimated to be 1.2 to 2.1 million patients, with approximately 425,000 patients having moderate or severe IMR with heart failure. Such patients with class II or worse IMR receiving current surgical treatment have a poor prognosis, with a five years survival of only about 50%. Implanting an undersized mitral annuloplasty ring or an unusually shaped mitral annuloplasty ring may, when tenting is minimal, temporarily mitigate mitral regurgitation, but after a few months IMR often returns and progresses. The failure of the use of a mitral annuloplasty ring alone, or in conjunction with coronary artery bypass grafting, is because the root cause of the problem is ventricular, not annular in nature. While coronary artery bypass grafting helps prevent further infarctions it does not significantly address the already damaged portion of the left ventricle. In patients with IMR the valve annulus may be normal, and the leaflets may be normal, and the chordae tendineae may be normal, and sometimes even the papillary muscles may be normal but severe mitral regurgitation is present because the leaflets cannot coapt due to ventricular distortions.

Current surgical methods of treating IMR include one or more of the following: coronary artery bypass grafting surgery; implanting undersized or unusually shaped annuloplasty rings (discussed above); implanting a cloth band around the opposing papillary muscles to draw and retain the distance between them to near normal; and severing secondary chordae tendineae. Another method that has been tried is to use a suture between the papillary muscle and the annulus in an attempt to raise the papillary muscle relative to the annulus. However, the suture may pull out of the tissue or abrade the ventricular wall. The implantation of a prosthetic or bioprosthetic mitral valve is sometimes used to replace the healthy but regurgitant valve and thus treats the mitral regurgitation. However this technique does not address the ventricular disorder. Thus in spite of surgical intervention ventricular dysfunction often increases as the ventricle further dilates.

Various embodiments of the invention disclosed herein overcome at least some of the drawbacks of current methods of treatment by addressing the ventricular disorder while sparing the natural mitral valve without over restriction of the valve area.

### SUMMARY OF THE EMBODIMENTS

One embodiment is a method for the treatment of mitral regurgitation of a damaged left ventricle of a heart, the left ventricle having at least one of first and second opposing papillary muscles displaced laterally or axially relative to the mitral annulus as compared to normal lateral and axial positions of the opposing papillary muscles relative to the mitral annulus of a healthy heart. The method comprises halting of the beating heart and extending a segment of a first lateral limb across opposing sides of the ventricle at locations whereby the first and second opposing papillary muscles may be drawn together laterally by shortening the segment of the first lateral limb. The segment of the first lateral limb is then shortened as needed to put the at least one of the first and second opposing papillary muscles into a substantially normal lateral position relative to the mitral annulus. The length of the segment of the first lateral limb is then fixed. A segment of a first axial limb is extended between the first lateral limb near a first opposing papillary muscle and the mitral annulus. A segment of a second axial limb is extended between the first lateral limb near a second opposing papillary muscle and the mitral annulus. The segments of the first and second axial limbs are shortened as needed to put the at least one of the first and second opposing papillary muscles into a substantially normal axial position relative to the mitral annulus and the length of the segments of the first and second axial limbs are fixed.

This embodiment may further include the first lateral limb protruding through the ventricle wall to outside the ventricle and the shortening of the first lateral limb may be performed by moving the ventricle wall inward. The fixing step may be performed by securing the at least one end of the first lateral limb to an outside surface of the ventricle wall. This embodiment may further include resuming the beating of the heart and then adjusting the length of the first lateral limb by un-securing the at least one end of the first lateral limb from the outside surface of the ventricle wall and moving the ventricle wall inward or outward, as desired, and re-securing the at least one end of the first lateral limb to the outside surface of the ventricle wall.

This embodiment may also include attaching a first line to the segment of the first axial limb at a point spaced from the first lateral limb and extending the line axially of the first axial limb opposite the mitral annulus through the wall of the ventricle substantially opposite the mitral annulus to outside the ventricle. In a similar manner, a second line is attached to the second axial limb. The first and second lines are then secured on an outside surface of the ventricle wall. The beating of the heart may be resumed and the length of the segment of the first lateral limb may be adjusted as needed by un-securing the at least one end of the first lateral limb from the outside surface of the ventricle wall, moving the ventricle wall inward or outward, as needed, and re-securing the at least one end of the first lateral limb to the outside surface of the heart. The limb segments of the first axial limb and the second axial limb may also be adjusted, as needed, by un-securing the first or second lines from the outside surface of the ventricle wall and drawing the first and second lines into or out of the ventricle to lengthen or shorten the segment of the first and second axial limbs, respectively, as needed, and re-securing the first or second lines on the outside surface of the ventricle wall.

Another embodiment is a device for the treatment of schemic mitral regurgitation of a damaged left ventricle having a first and second opposing papillary muscles displaced laterally or axially relative to a mitral annulus as compared to a normal lateral and axial position of opposing papillary muscles relative to a mitral annulus of a healthy heart. The device comprises a first lateral limb configured to extend between opposing papillary muscles of a left ventricle and first and second axial limbs extending from spaced points on the lateral limb to form an essentially U-shaped configuration. The first and second axial limbs are configured to extend between the lateral limb and the mitral annulus. The lateral limb and the first and second axial limbs may further comprise a biocompatible, axially compressible housing extending axially of each limb. The device may further comprise the lateral limb further comprising a folded suture defining a drawstring pair joined to the lateral housing at the suture fold near an axial limb with free ends of the drawstring pair extending form the housing. The device may further comprise the first and second axial limbs each comprising folded suture defining a first drawstring pair joined to the axial limb housing at the suture fold near the lateral limb with free ends of the drawstring pair extending axially from a distal end of the axial housing. The first pair of drawstrings may each have an axle operatively associated therewith at a point within the axial housing intermediate a proximal end of housing in a distal end of the housing. The first and second axial limbs may further comprise second folded sutures defining a second drawstring pair, the second folded sutures being draped over the axle with the free ends of the second drawstring pairs extending axially from the proximal ends of the axial housing.

Another embodiment comprises a U-shaped or “Trapeze” shaped body member or housing having a lateral limb and two or more axial limbs. The limbs comprise a housing that may be of a fixed length or may be contractible. The axial limb housings may be of equal length, or one limb housing may be longer than the other. The axial limbs are separated by a lower lateral limb. In an embodiment where the limb housings are contractible the axial limb housings have a length in the approximate range of 25 mm to 50 mm in un-contracted length, and the lateral limb housing has a length of approximately 75 mm in un-contracted length, but the actual dimensions depend in part on the size of the heart into which the device is to be implanted. It may be necessary to have more than one size available for surgical use. The housing may be a hollow braided Polyester tubular form and preferably has small loops terminating the upper ends of the axial members. The body may further comprise three drawstring loops, one in each axial limb housing, and the third in the lateral limb housing. The drawstring loops in each of the first and second axial limb housings may be anchored near the bottom of its respective limb housing and the free ends or tails of the drawstring loop emerges at the apex of the respective loop in the axial limb housing. The lateral limb, having a first end and a second end, will be placed in the left ventricle at the approximate level of the first and second papillary muscles, though it may be at the base of the papillary muscles. The drawstring loop in the lateral limb housing may be anchored near to the first end of the lateral body and adjacent to the bottom of first axial limb, and the two tails of the drawstring loop may be passed through the body wall and emerge near the second end of the lateral portion and adjacent to the bottom of the second axial limb. The first end of the lateral body is sewn or otherwise attached to the papillary muscles by a suture passed through the papillary muscle. The drawstring tails of the lateral portion of the device may emanate from the lateral limb and be shortened from within the ventricular cavity. In an alternative embodiment, the lateral drawstrings are passed through the ventricular wall near a papillary muscle to emerge from the endocardium. These drawstrings are secured to the outside wall of the ventricle, typically by means of a pledget, and may be tied or clipped to the pledget. The drawstrings associated with the first axial limb are passed upwards through the mitral annulus into the left atrium approximately directly above and in line with the first papillary muscle, and likewise the drawstrings associated with the second axial limb are passed upwards through the mitral annulus into the left atrium approximately directly in line with the second papillary muscle. Following implantation the lateral drawstrings are tightened to draw the papillary muscles inwardly towards each other until they are a near normal distance apart. Likewise the drawstrings associated with each axial limb are tightened to pull the papillary muscles axially towards the mitral annulus until near normal distances are achieved. These maneuvers eliminate the “tenting” and allow the mitral valve leaflets to coapt correctly.

In the embodiment where the lateral drawstring tails are passed through the ventricular wall the drawstrings may have cardiovascular surgical needles attached to their distal ends, and the suture is passed through the ventricular wall near a papillary muscle.

In one embodiment of the invention the two pairs of drawstrings from the axial limbs are also passed through the sewing ring portion of a mitral annuloplasty ring implanted on the atrial aspect of the mitral annulus, and following suitable adjustment are tied off and the excess drawstrings are cut off. The annuloplasty ring or band may be a rigid structure in the lateral and posterior segments. The purpose of the annuloplasty ring is to both fix and stabilize the dimensions and shape of the annulus, and also to act as a supporting strut for the subvalvular component of the device, and hence spread the vertical axial load around the annulus.

In an embodiment of the invention the sub-valvular housing is composed of a single braided axially collapsible Polyester tube containing at least three pairs of drawstrings. The polyester tube may be heat set in a U-shaped or trapeze configuration. Alternatively, two or three lengths of braided tubing joined together could be used. The collapsible tube or tubes could be made of other suitable biocompatible material such as PTFE. The braided tube may have loops formed or sewn at each end of the axial limb housing, from each of which two distal ends of a drawstrings emerge. The distal ends of the drawstrings are passed through the braided tube and each drawstring passes through an opposing position of the loop to space the drawstrings and to form anchor points.

In one embodiment a hollow thin wall tube that may include a removable trochar is pushed through the mitral annulus at the appropriate point above the papillary muscles. Upon removal of the trochar a loop of a thin flexible wire loop (which may be a self expanding super-elastic Nitinol wire loop) is pushed through the tube into the left ventricle. The drawstring tail of an aligned axial limb is threaded into the wire loop, and the wire loop is then pulled back into the left atrium bringing the drawstring with it. The other axial limb drawstring tails are likewise pulled into the left atrium. These drawstring tails may be passed through a sewing cushion of the annuloplasty ring.

In another embodiment, tabs, which may be made of a biocompatible fabric, are attached to the bottom of the axial limbs near the lateral limb. The tabs are configured to wrap around an adjacent papillary muscle during implanting of the device, and each tab may be stitched into a loop sutured to the papillary muscle. The tabs help prevent the sutures attaching the device to the papillary muscles from being torn from the papillary muscles.

The various embodiments may be employed in any combination recognized as appropriate to one of skill in the art. Discussion as “another” embodiment or “one” embodiment does not necessarily mean alternatively or distinct from other embodiments discussed herein. As used herein “or” is intended to mean and/or and not singular alternatives unless specified.

### BRIEF DESCRIPTION OF THE DRAWINGS

FIG. 1 is a schematic sectional view through a left ventricle of a heart with ischemic (functional) mitral regurgitation showing tenting of the leaflets and central mitral regurgitation;

FIG. 2 is a front elevation view of an embodiment of a device employing drawstrings for contracting the lateral and axial limb segments for treatment of ischemic mitral regurgitation;

FIG. 3 is a side elevation of a distal end of an axial limb segment of FIG. 2 taken in the direction of arrow B in FIG. 2;

FIG. 4 is a cross-section view taken along line CC of FIG. 2;

FIG. 5 is a cross-section view taken along line DD of FIG. 2;

FIG. 6 is a cross-section view taken along line EE of FIG. 2;

FIG. 7 is a schematic sectional view through a left ventricle of a heart with functional mitral regurgitation and with the an embodiment of device employing drawstrings for contracting segments of the lateral and axial limbs of FIG. 2 implanted in the ventricle prior to tightening and tying of the drawstrings and suture;

FIG. 8 is a schematic sectional view through a left ventricle of a heart with prior functional mitral regurgitation with the an embodiment of device employing drawstrings for contracting the lateral and axial limbs segments of FIG. 2 implanted in the ventricle and an annuloplasty ring implanted on the mitral annulus following tightening and tying of the drawstrings;

FIG. 9 is a front elevation view of an embodiment of a device not employing drawstrings for contracting the lateral and axial limbs for treatment of ischemic mitral regurgitation;

FIG. 10 is a schematic sectional view through a left ventricle of a heart with prior functional mitral regurgitation with the an embodiment of device not employing drawstrings for contracting the lateral and axial limbs of FIG. 9 implanted in the ventricle and an annuloplasty ring implanted on the mitral annulus;

FIG. 11 is a front elevation view of another embodiment of a device employing drawstrings for contracting the lateral and axial limb segments for treatment of ischemic mitral regurgitation having more than two axial limbs;

FIG. 12 is a side elevation of the right axial limbs of the embodiment of FIG. 11 taken in the direction of the arrow B of FIG. 11;

FIG. 13 is a side view of a tubular casing for inserting an axial drawstring through the mitral annulus;

FIG. 14 is a cross-section of the tubular casing of FIG. 13 taken along line 14-14 of FIG. 13;

FIG. 15 is a side elevation of a trochar for use with the tubular casing shown in FIG. 13;

FIG. 16 is a side elevation of a loop member for use with the tubular casing shown in FIG. 13;

FIG. 17 is a side view of a tubular casing shown in FIG. 14 with the trochar member shown in FIG. 15 fully inserted into the tube;

FIG. 18 is a side view of a tubular casing shown in FIG. 14 with the trochar member removed and the loop member shown in FIG. 16 fully inserted into the tube;

FIG. 19 is a perspective view of an embodiment of a device similar to that depicted in FIG. 2 but further including tabs attached to the axial limbs near the lateral limb;

FIG. 20 is a schematic perspective view of the device of FIG. 19 shown with the tabs wrapped around and sutured to the papillary muscles of a left ventricle of a heart;

FIG. 21 is a perspective view of an embodiment of a device similar to that depicted in FIG. 19, further including drawstring pairs extending axially from a proximal end of the axial limbs near the lateral limb;

FIG. 22 is a cross-section of the device of FIG. 21;

FIG. 23 is an enlargement of an attachment of a second drawstring pair or a first drawstring as depicted in FIG. 22;

FIG. 24 is a front elevation view of a further embodiment of a device for the treatment of mitral regurgitation;

FIG. 25 is a schematic sectional view through a left ventricle of a heart with functional mitral regurgitation with the device of FIG. 24 implanted in the ventricle prior to tightening and tying of the drawstrings;

FIG. 26 is a schematic sectional view through a left ventricle of a heart with prior functional mitral regurgitation with the device of FIG. 24 implanted in the ventricle and a annuloplasty ring implanted on the mitral annulus following tightening and tying of the drawstrings;

FIG. 27 is a schematic sectional plan view of a left ventricle of a heart showing an embodiment of the device of FIG. 24 having three lateral limbs formed in a triangle; and

FIG. 28 is a schematic sectional view through a left ventricle of a heart depicting a further modified embodiment of the device FIG. 27 including three axial limb extending near the vertices of the triangle formed by the three lateral limbs.

### DETAILED DESCRIPTION

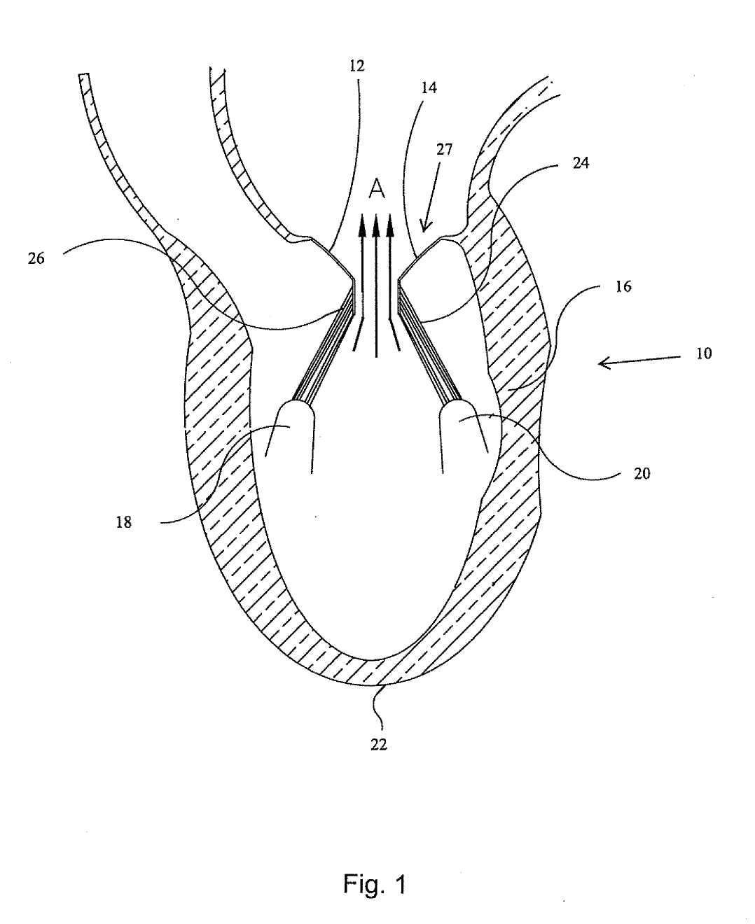

FIG. 1 is a schematic simplified cross-sectional view through the left ventricle of a heart 10 with functional mitral regurgitation in systole, showing tenting of the leaflets 12, 14 and central mitral regurgitation jet depicted by three arrows labeled “A”. The dilatation of the thinned section of the left ventricular wall illustrated at 16 has caused the papillary muscles 18, 20 to move outwardly and downwardly toward the ventricular apex 22, causing chordae tendineae 24, 26 to pull leaflets 12, 14 apart allowing a central regurgitant flow, shown by the three arrows labeled “A”, between the now non-coaptive areas of the leaflets 12, 14.

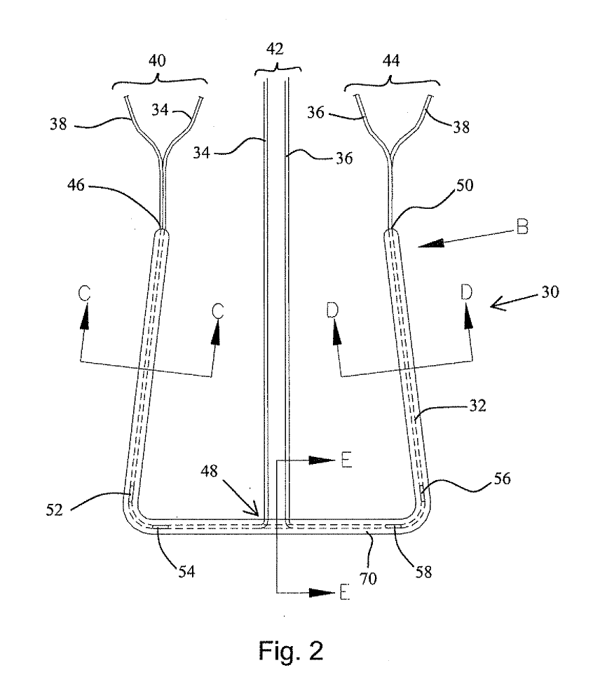

An embodiment of the invention shown in FIGS. 2-6 comprises a ventricular device 30 comprising a pair of axial limbs joined to and separated by a lateral limb. The limbs comprise a flexible and collapsible housing 32 which in the illustrated embodiment is made from a single length of braided Polyester tube of approximately 2.5 mm outside diameter and approximately 1.25 mm internal diameter. A hollow braided Polyester tube used in the construction of the device has several advantages. For example it has the ability to readily be axially compressed without buckling, it retrains and confines the active portions of the drawstring and prevents them from tangling with the papillary muscles or chordae tendineae. It also protects the tissue of the heart from being abraded by the drawstrings. Other housing materials sharing the properties may also be used in the practice of the invention. For example, the drawstrings might be coated with a suitable biocompatible plastic.

The flexible, collapsible housing 32 contains three drawstrings formed of lengths of surgical suture material 34, 36, 38 that may be of size 2 braided Polyester surgical suture of two distinct colors for easy identification. For example, the drawstrings 34, 36 may be white and the drawstring 38 may be green. These three lengths form three sets of drawstring pairs 40, 42, 44 as shown in FIG. 2. The drawstring pairs above may define a pair of axial limbs and lateral limb there between, and the limbs may include the housing as illustrated herein. The drawstring pairs 40, 42, 44 exit the tubular braid at 46, 48, 50 respectively. The length of suture material 34 is anchored inside the tubular braid at 52 and 54. The length of suture material 36 is anchored at 56 and 58. The length of suture material 38 is anchored at 52 and 56. The anchors 52, 54, 56, 58 may be accomplished by an suitable means for securing the point of attachment of the sutures to the flexible and collapsible housing 32 so that the point of attachment does not move relative to the flexible and collapsible housing 32. Suitable means of attachment may be clips, staples, anchors, sutures and other structures or means such as adhesives or heat staking known in the art made of biocompatible materials.

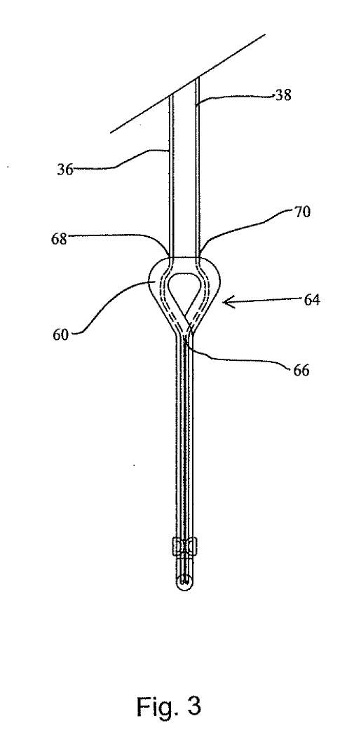

FIG. 3 shows a distal end of one of the two upright axial limb housings of the device 30 in the direction of arrow B of FIG. 2. A loop 60 is shown in a generally triangular form, but the actual form is relatively unimportant as the purpose of the loop is to separate the two drawstring tails 36, 38 (and likewise 34, 38). The loop 60 is formed by folding back a distal end 64 of the tubular housing 32 and sewing or otherwise joining to the housing 32 at point 66. Drawstring tail 36 emerges at point 68 and drawstring tail 38 emerges from the loop at point 70.

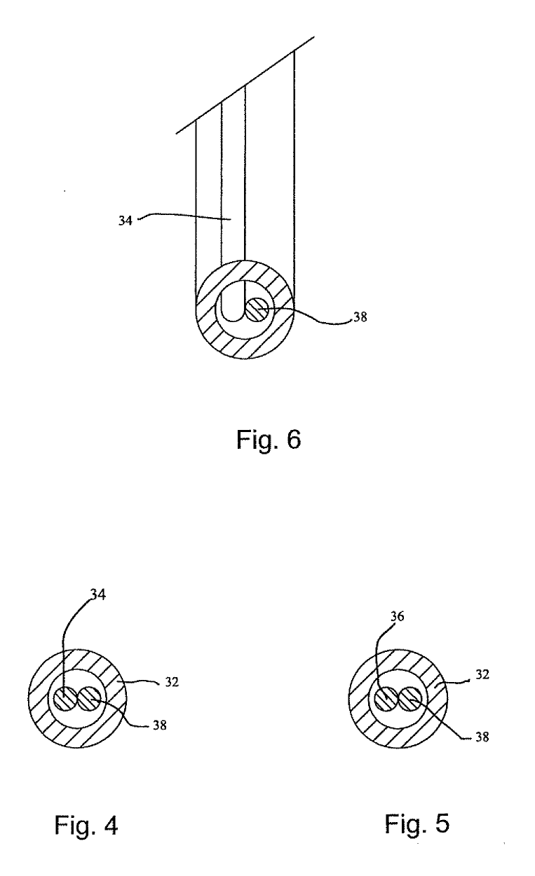

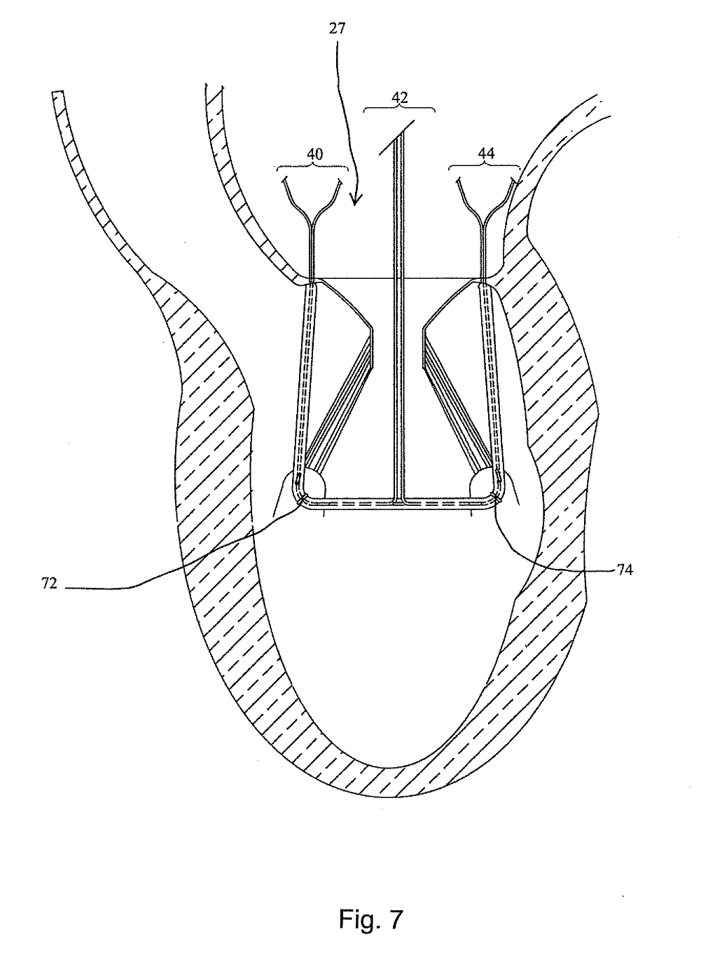

FIG. 4 shows a cross-sectional view taken along line CC of FIG. 2, illustrating the lengths of suture material 34, 38 defining the drawstring pair 40 and FIG. 5 shows a cross-sectional view taken along line DD of FIG. 2 illustrating the lengths of suture material 36, 38 defining drawstring pair 44. FIG. 6 shows a cross-sectional view taken along line EE of FIG. 2 illustrating length of suture material 36, which is one half of drawstring pair 42.

In an embodiment where the flexible and collapsible housing 32 is a polyester tube, it may be heat set (or folded) into a generally “U” or “Trapeze” shaped form as shown in FIG. 2. When implanted the bottom lateral limb 70 of the device 30 lies in a substantially straight line between the opposing papillary muscles. The lateral drawstrings 34, 36 of drawstring pair 42 passes though the lateral limb with the drawstring anchored to the polyester tube at 54, 58, respectively, near the first ends of the lateral portion of the housing that lies against and are sewn to the papillary muscle by means of cardiovascular sutures 72, 74 as illustrated in FIG. 7. Alternatively, one or both of the ends of the lateral portions of the housing could be sewn to the interior or exterior wall of the ventricle if deemed appropriate by the physician. If secured to the exterior wall, anchors or pledgets may be attached to the exterior walls of the ventricle with the surgical sutures 72, 74 attached thereto.

With the device implanted as shown in FIG. 7, or with the ends of the lateral portions sewn to the base of the papillary muscles or the interior or exterior of the ventricle wall, drawstring pairs 40, 44 are passed through the mitral annulus 27 and drawstring pair 42 is passed through the central area of the valve.

In the embodiment illustrated herein, a rigid mitral annuloplasty ring or band may be implanted at the same time as the device as the annuloplasty ring acts as a load bearing strut to distribute axial downwards pull exerted by drawstrings pairs 40, 44 and prevents unwanted dilation or distortion of the mitral annulus. Alternatively, other structures such as buttons, pledgets, ring segments (rigid or flexible) or a flexible or semi-rigid annuloplasty ring may be used if the annulus is healthy and not distorted, provided they can effectively distribute the axial loads without undue distortion of the mitral annulus. “Undue” means distortion interfering with the long term integrity of the mitral valve.

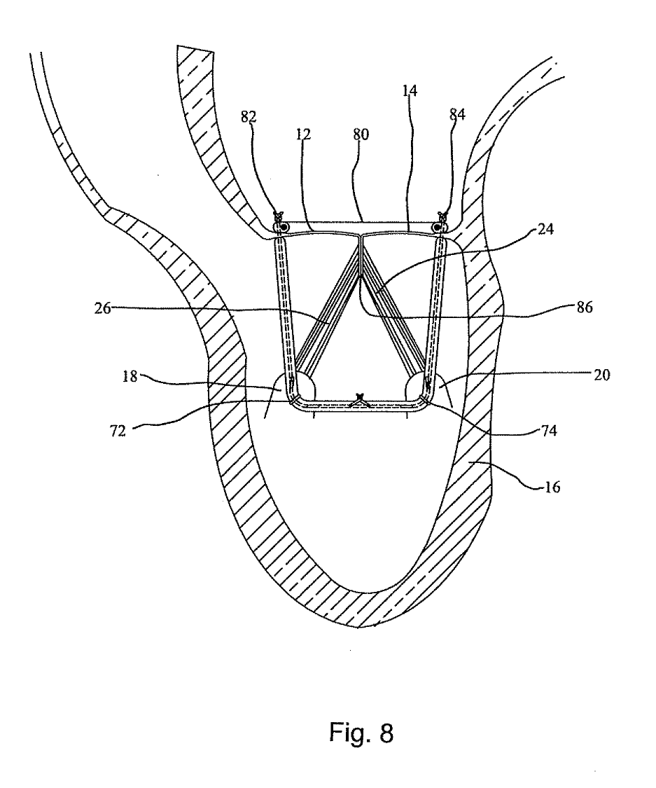

FIG. 8 shows the final implantation of a rigid annuloplasty ring 80 in conjunction with the device 30. However, prior to finalizing the implantation step the lateral drawstring pair 42 is tightened (an appropriate amount to draw the two papillary muscles laterally towards each so that they are separated a distance present in a healthy heart (i.e., a “normal” distance). Likewise the other two drawstring pairs 40, 44 are tightened to draw the papillary muscles axially towards the mitral annulus until near normal distances between the apexes of the papillary muscles and the mitral annulus are achieved. Following the initial tightening of the drawstrings it is prudent to test of competency of the valve by injecting liquid, such as physiological saline solution, into the left ventricle at near physiological pressure to demonstrate that the valve leakage has been corrected. Finally the three sets of drawstrings are tied off and trimmed to length.

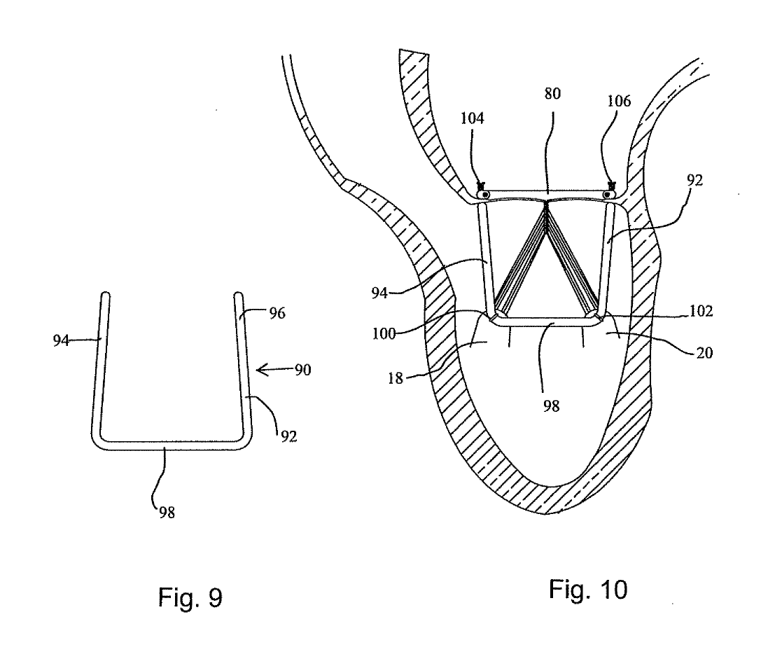

The normal implantation sutures used to complete the attachment of the annuloplasty ring to the annulus are not shown. Drawstring pairs 40 and 44 are terminated in knots 82, 84 abutting the annuloplasty ring 80. It may be seen in FIG. 9 that the leaflets now coapt correctly at 86. The operation may be completed in the normal manner, in conjunction with coronary artery bypass grafting, if appropriate. An advantage of this structure is the fit can be customized to restore the papillary muscles to their normal position in hearts of varying dimensions. The device 30 (as well as all embodiments of the devices described herein) may also be installed robotically.

FIG. 9 is a front elevation view of an embodiment of device not employing drawstrings for contracting the lateral and axial limbs for treatment of ischemic mitral regurgitation. In this embodiment device 90 comprises a housing 92 which is generally U-shaped in configuration and comprises a first axial limb 94 and a second axial limb 96 joined by a lateral limb 98. In this embodiment the lengths of the axial limbs 94, 96 and the lateral limb 98 are not adjustable by the use of drawstrings and thus the device 90 must be dimensioned to restore the papillary muscles to a normal position as illustrated in FIG. 10. Some measure of adjustability can be provided whereby the surgeon attaches the junction between the lateral limb 98 and the axial limbs 92, 94 to the papillary muscles 18, 20. For example, in FIG. 10, these junctions are joined by surgical sutures 100, 102 near a distal end of the papillary muscles 18, 20. Alternatively, if deemed appropriate by the physician, these junctions could be attached by the sutures 100, 102 proximal or at the base of the papillary muscles 18, 20 or even directly to the interior sidewalls of the ventricle or through the ventricle side wall to an outer surface as required by the heart anatomy of a particular patient. The distal ends of the axial limbs 92, 94 are attached to an annuloplasty ring 80 by sutures 104, 106 as illustrated in FIG. 10.

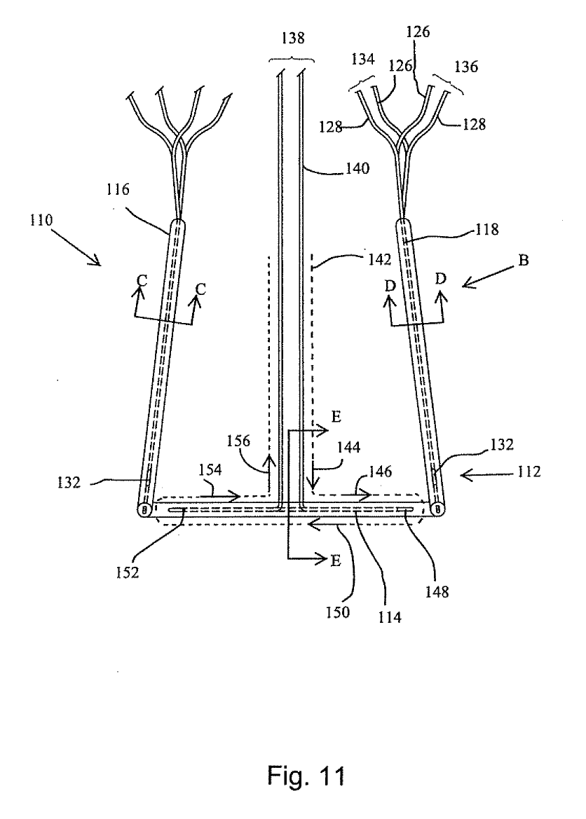

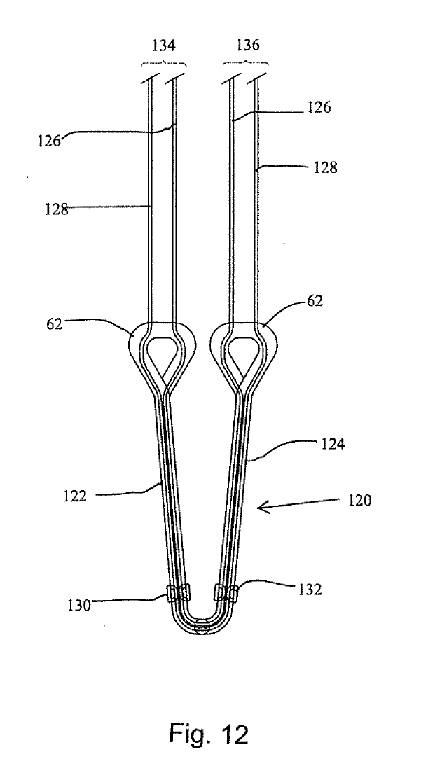

Yet another embodiment of the ventricular device for mitral valve regurgitation 110 is illustrated in FIG. 11. The most significant deviation of the embodiment 110 is that it features two axial limbs at each end of the lateral limb. More particularly, the housing 112 which may be formed of a heat set braided polyester as is the case with the embodiment 30 illustrated in FIG. 10, consists of a lateral housing limb 114 having a pair of axial housing limbs 116 at one distal end and a second pair of axial housing limbs 118 at a second distal end. For example, the lateral limb 114 may be formed of one segment of a heat set, braidable, biocompatible material 114 and the first and second pairs of axial limbs 116, 118 can be formed of another segment of the same material heat set into a V-configuration 120, as illustrated in FIG. 12. The lateral housing limb 114 and the first and second axial housing limbs 116, 118 formed into the V as illustrated in FIGS. 11 and 12 can then be sutured, heat set, glued, staked or otherwise attached together to form a single unit.

Referring to FIGS. 11-12, each pair 116, 118 of axial housing limbs forms a V-configuration 120. Distal ends of each of the axial housing limbs 122, 124 are formed into a loop in the same manner and for the same purpose as the loops 62 illustrated in FIG. 3. Each of the first and second axial pairs 116, 118 include a pair of drawstrings 134, 136 for contracting the first and second axial housing limbs 122, 124 and for securing the axial limbs 122, 124 to the mitral annulus or an annuloplasty ring in the same manner the axial limbs of the embodiment 32 are attached to the annuloplasty ring as discussed with reference to FIGS. 7 and 8. In the embodiment 110, a first length of suture material 126 is fed through the V-shaped housing 120 and a second length of suture material 128 is likewise fed through the housing material with two of the lengths of suture materials secured by anchors 130, 132 within the V-shaped housing 120. In this manner the ends of the first length of suture material 126 and second length of suture material 128 form a first drawstring pair 134 and a second drawstring pair 136.

A lateral drawstring pair 138 is defined by a length of suture material 140 which has its ends extending from the lateral limb 114 as illustrated in FIG. 11. This length of suture material 140 is directed through and out the lateral limb 114 as illustrated by the ghost lines 142 in FIG. 11. More particularly, the length of suture material 140 is fed from a center of the lateral limb 114 illustrated at arrow 144 to the right as illustrated at arrow 146 around and fastened to the anchor 148, to the left as illustrated by arrow 150 around and fastened to the anchor 152 to the right as illustrated at arrow 154 and out of the center of the lateral housing as illustrated by the arrow 156.

The embodiment 110 is installed in the heart in virtually the same manner as discussed above with the embodiment 30 and as illustrated in FIGS. 7 and 8. The primary difference is with two pairs of axial limbs 116, 118, there can be four points of attachment of axial limbs to the annuloplasty ring 80 to provide more axial support. Attachment is made by attaching the drawstring pairs 134, 136 to the annuloplasty ring. The points of attachment to the annuloplasty ring can be decided by the surgeon. For example, two points of attachment may be at about the commissural cusps above the papillary muscles and the other points of attachment may be anterior and posterior to the commissural cusps near or approaching the trigones. As should be readily apparent, the axial length of each axial limb housing segment 122, 124 can be adjusted to raise the papillary muscle to a normal position by tightening the drawstring pairs 134, 136 and then tying the length of suture materials 126, 128 of each drawstring pair 134, 136 together. The lateral distance between the papillary muscles can be shortened to return them to a normal position by tightening the drawstring pair 138 and tying them into a knot in a similar manner discussed in regard to the drawstring pair 42 in the embodiment 30 of FIG. 2. It may be useful or desirable to have some instrument to hold the lateral limb 114 down while tightening the drawstring pair 138, as may also be the case with regard to the drawstring pair 42 in the embodiment 30 of FIG. 2.

As discussed above, having the four axial limbs allows for four points of attachment to the annuloplasty ring. More or less limbs could be provided for more or less points of attachment as deemed necessary. More than four could complicate installation. Thus, providing three or four axial limbs may be the most desirable though this is not yet determined with certainty.

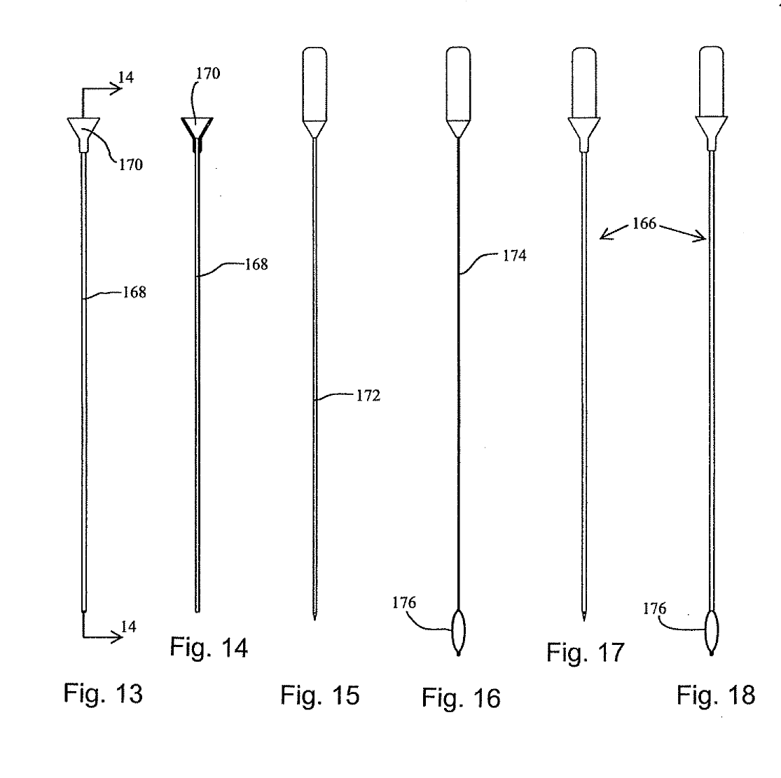

The difficulty of placing the axial drawstrings correctly through the mitral annulus approximately in line with the papillary muscles is addressed by the implanting device 166 in FIG. 13 through FIG. 18. FIG. 13 is a side view of a tubular casing 168 for inserting an axial drawstring through the mitral annulus having a funnel 170 at its proximal end. FIG. 14 shows a cross-section of FIG. 13 along line 14-14 of FIG. 13. FIG. 15 is a side elevation of a trochar 172 for use with the tubular casing 168 shown in FIG. 13. FIG. 16 is a side elevation of a loop member 174 for use with the tubular housing 16 shown in FIG. 13. FIG. 17 is a side view of a tube housing 168 shown in FIG. 13 with the trochar 172 fully inserted into the tube. FIG. 18 is a side view of the tubular housing 168 with the trochar 172 removed and the loop member 174 fully inserted into the tube 168.

In use the trochar 172 is fully inserted into the tube 168 as shown in FIG. 17. The assembly is the pushed through the mitral annulus to project only a few millimeters into the ventricular cavity. The trochar 172 is removed from the tube 168. The tube is then carefully advanced into the ventricle to below the level of the valve leaflets. Loop member 174 is the introduced into the funnel 170 of the tube 168 and advanced through the tube to emerge at the lower end in the ventricle. On emerging from the tube the loop 176 will reform due to the springiness of the loop material (that could be of 0.009″ diameter Nitinol or stainless steel wire, or Nylon monofilament). The free end of one drawstring is then passed through the loop. The loop is then pulled back into the left atrium bringing the drawstring with it. Finally, the tube 168 is removed.

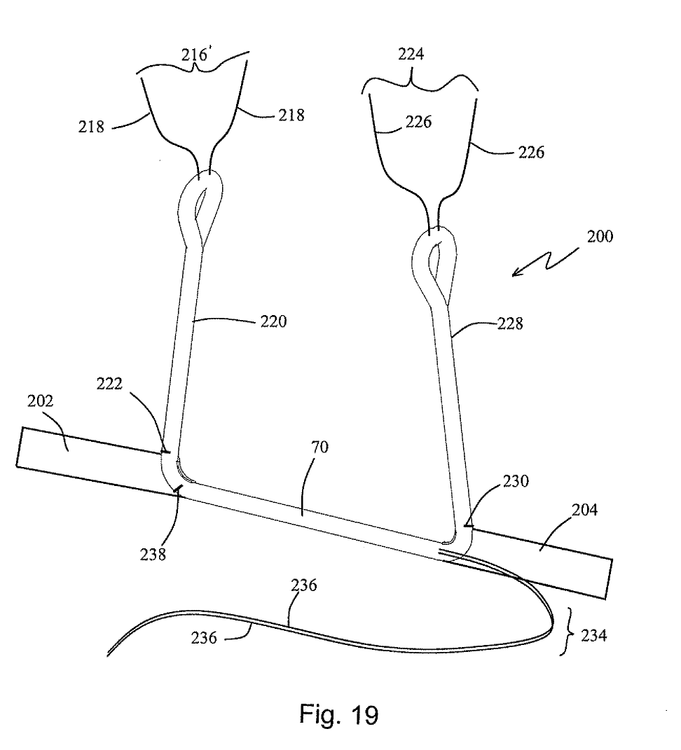

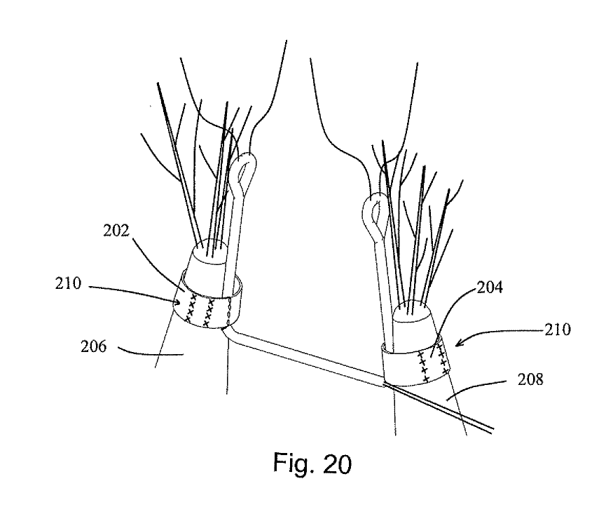

A further embodiment of the ventricular device for mitral valve regurgitation 200 is illustrated in FIG. 19. In one embodiment, which is not shown, the further embodiment of the ventricular device is essentially the same as that depicted in FIGS. 2-7, differing only in the inclusion of tabs 202, 204 attached near the bottom of the axial limbs proximate the lateral limb 70, as illustrated in the embodiment 200 of FIG. 19. As depicted in FIG. 20, the tabs 202, 204 are configured to wrap around a papillary muscle adjacent thereto. The tabs 202, 204 may be made of a biocompatible fabric. As depicted in FIG. 20, in use, the tabs 202, 204 wrap around the papillary muscles 206, 208 and the tab is sewn to itself around the papillary muscles. Sutures 210 are implanted through the tabs and into the papillary muscle to secure the ventricular device 200 into place. When used with a ventricular device 30 of the type depicted in FIG. 2, the device is otherwise installed in the manner discussed above with regard to the embodiment 30.

The particular embodiment 200 illustrated in FIG. 19 differs from the embodiment 30 in that the pair of drawstrings 216 consist of a single length of suture 218 which is folded and attached to the axial limb housing 220 near the lateral limb 70 at the fold by the anchor 222. In a like manner the pair of drawstrings 224 consists of a single length of suture 226 which is folded and attached to the axial limb housing 228 by the anchor 230 near the lateral limb 70 at the suture fold. The third pair of drawstrings 234 consists of a length of suture 236 that is folded and attached to the lateral limb housing 70 by the anchor 238 near the axial limb 220 at the suture fold. In this embodiment the drawstring pair 234 then extends axially out of the lateral limb housing 70 proximate the axial limb 228. The drawstring pair 232 may be passed through the wall of the ventricle and secured in cooperation with an anchor or pledget to the epicardium (outside wall of the ventricle) with the lateral limb segment within the ventricle of a desired length. Alternatively, the lateral suture pair 234 may be used to adjust the length of the lateral limb segment 70 and tied together inside the ventricle adjacent the papillary muscle 208. The embodiment 200 as depicted in FIG. 19 would otherwise be implanted in the same manner discussed above with regard to the embodiment 30.

The further embodiment of the ventricular device 200 benefits from the tabs 202, 204 acting as reinforcing members to prevent implantation sutures from tearing out of the papillary muscles, as shown in FIG. 20.

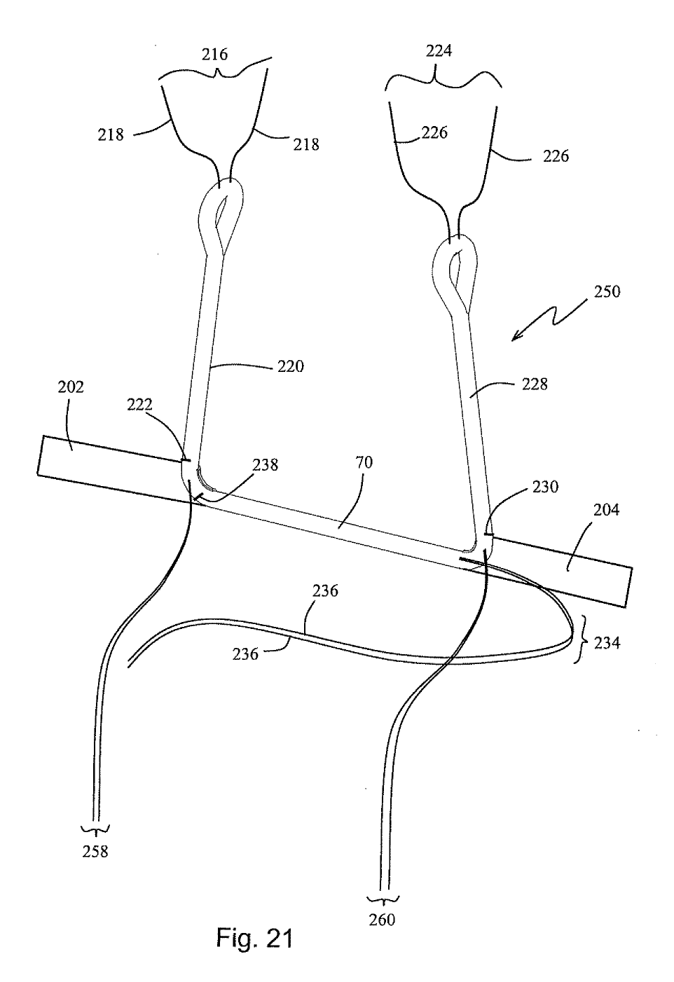

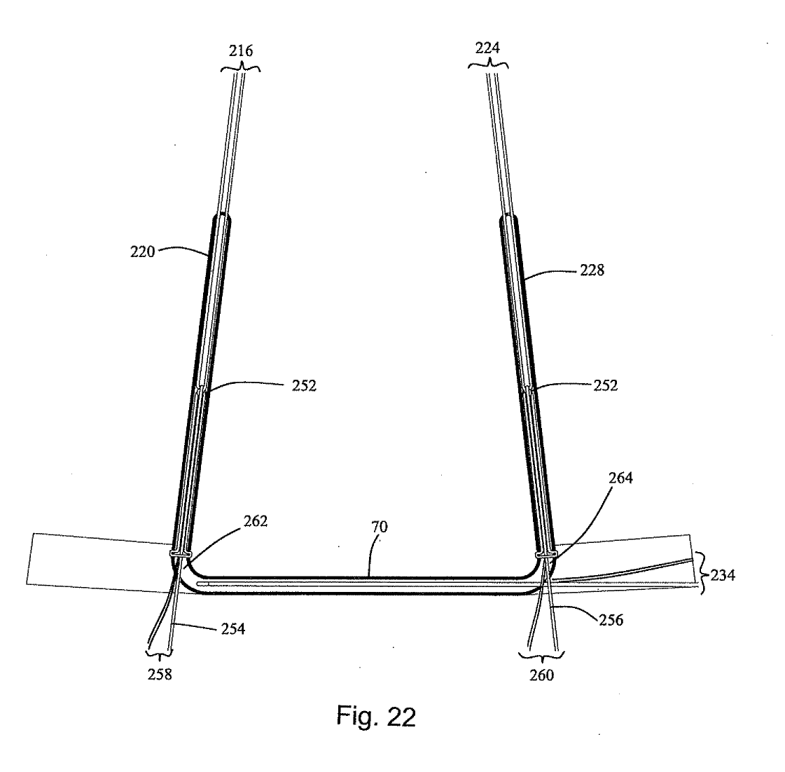

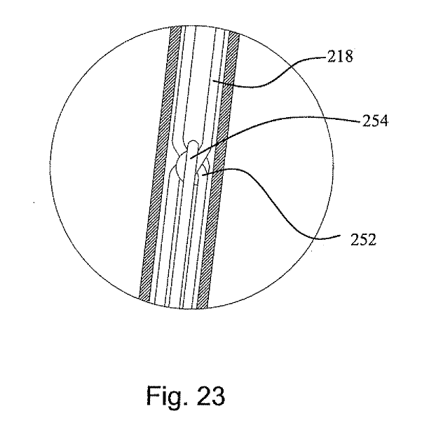

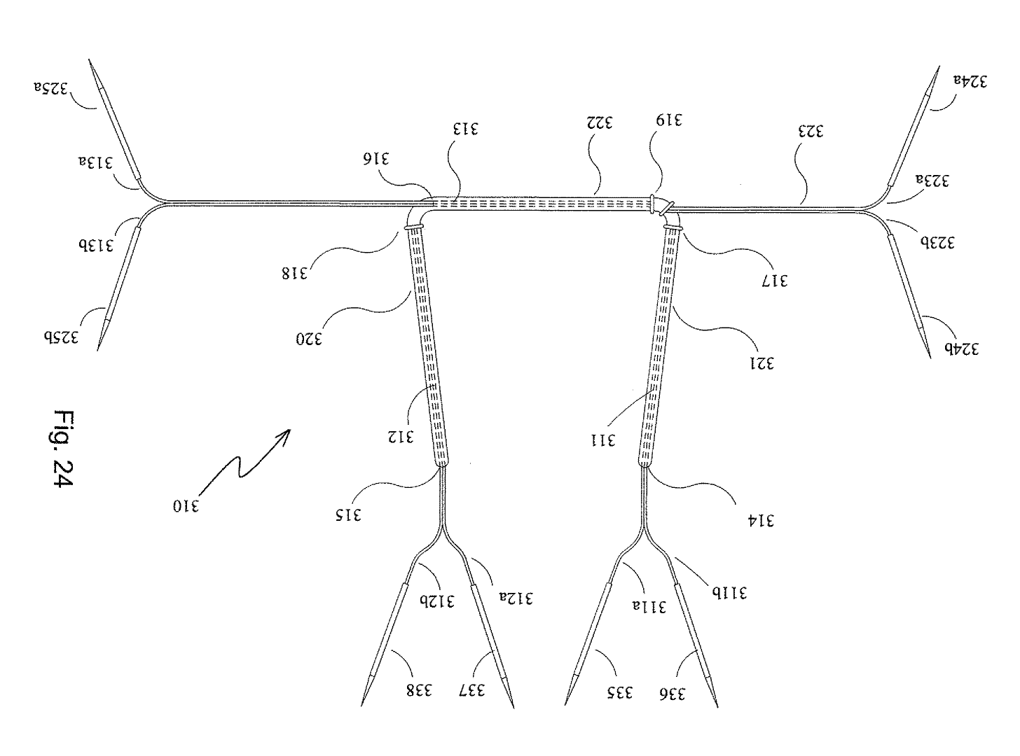

FIG. 21 is another embodiment of the ventricular device for mitro-valve regurgitation 250. This embodiment is similar in many respects to the embodiment 200 illustrated in FIG. 19 and common elements will use the same reference number. Referring to FIG. 22, the biggest difference is in the embodiment 250 the sutures 218 and 226 each have a knot 252 formed inside the axial limb housings 220, 228 at a point spaced from the lateral limb 70 intermediate proximal and distal ends of the axial limb housings 220, 228. The knots 252 define in essence an axle, over which sutures 254, 256 are draped. This is shown in greater detail in FIG. 23. While the embodiment 250 illustrated in FIGS. 21-23 uses the knot 252 to define the axle, other structures such as buttons, tabs or the like could be used to form the axle. The draped sutures 254, 256 form a pair of drawstrings 258, 260. The drawstring pairs 258, 260 extend axially of the first and second axial limb housings opposite the drawstring pairs 216, 224 (or, when installed, opposite a mitral annulus) and protrude axially from a proximal end 262, 264 of the axial limbs housings 220, 228 near the lateral limb 70. The embodiment 250 illustrated in FIG. 22 includes the tabs 202, 204, but these need not be included. As in other embodiments illustrated herein, needles could be included at the distal ends of the drawstring pairs 258, 260, 234 to assist in installation.

The embodiment 250 may be installed in the left ventricle of a heart in a similar manner that the embodiment 200 is installed as discussed above. The primary difference is the additional steps of passing the drawstring pairs 258, 260 through the wall of the ventricle and securing the drawstring pairs to the epicardium or the surface of the outside wall of the ventricle by, for example, tying the drawstrings to a pledget or other similar load distributing device. This feature enables further axial adjustment of the distance between the papillary muscles and the mitral annulus following installation of the embodiment 250 in the left ventricle, closure of the heart, and cecession of cardiopulmonary bypass, i.e., on a beating, functioning heart.

An advantage of the embodiment 250 is that the drawstring pairs 216, 258 and 224, 260 are cooperatively used to raise the papillary muscles toward the mitral annulus to a “normal” position. In other words, an amount estimated to promote coaption of the mitral valve leaflets 14 once the heart begins beating again. Once the papillary muscles are returned to a substantially normal position, the drawstring pair 216 is then fixed by being tied or otherwise secured to the mitral annulus (or more commonly a load-bearing strut such as an annuloplastry ring) and the ends are cut. The drawstring pair 258 is tied to the pledget on the outside surface of the ventricle and the drawstring ends are cut in a manner leaving a length of the drawstrings at the pledget. In a like manner, the drawstring pairs 224, 260 are attached to secure the second axial limb 228 and reposition the papillary muscles. Likewise, the drawstring pair 234 are drawn to bring the papillary muscles to a substantially normal position further promoting coaption of the leaflet valves 14. This “drawing” may involve pushing the ventricle wall inward at the protruding drawstring. Drawstring pair 234 is then tied and cut leaving a length extending from the outside wall of the ventricle. The principle advantage of the embodiment 250 is that once the embodiment 250 is installed as described above, the drawstring pairs 258, 260 and 234 can be accessed outside the ventricle to allow for further manipulation of the position of the papillary muscles 268 relative to the mitral annulus. This may be desired when what was believed to be a “normal” positioning of the papillary muscles relative to the mitral annulus does not actually promote adequate coaption of the valve leaflets 14. In such a case, the drawstring pairs 258, 260 and 234 can be drawn tighter or released, as necessary, to tighten or lengthen the housing segments of the lateral and axial limbs within the ventricle to optimize the position of the papillary muscles to minimize or eliminate mitral regurgitation.

For example, after installation of the embodiment 250, after the heart is closed, the heart beat is restarted and the patient is weaned off cardiopulmonary bipass, an echo-cardio graph can be taken of the beating heart to determine whether there is any sign of mitral regurgitation. If so, further axial adjustment of the papillary muscles can be made by drawing tighter or releasing the drawstring pairs 258, 260 and re-securing them to their respective pledgets. Similarly, the lateral position of the papillary muscles can be adjusted by tightening or lengthening the lateral limb 70 by drawing out or releasing somewhat the drawstring pair 234 and re-securing the drawstring pair 234 to the associated pledget. If necessary, further echo-cardio graphs can be taken and further readjustment of the position of the papillary muscles relative to the mitral annulus can be made. It should be noted that the drawstring pairs 258, 260 and 234 can be secured to the outside wall of the ventricle tying a knot to a pledget or by the use of clips or any other suitable bio-compatible fastener to allow for ready adjustment of the length of the drawstring pairs as described above.

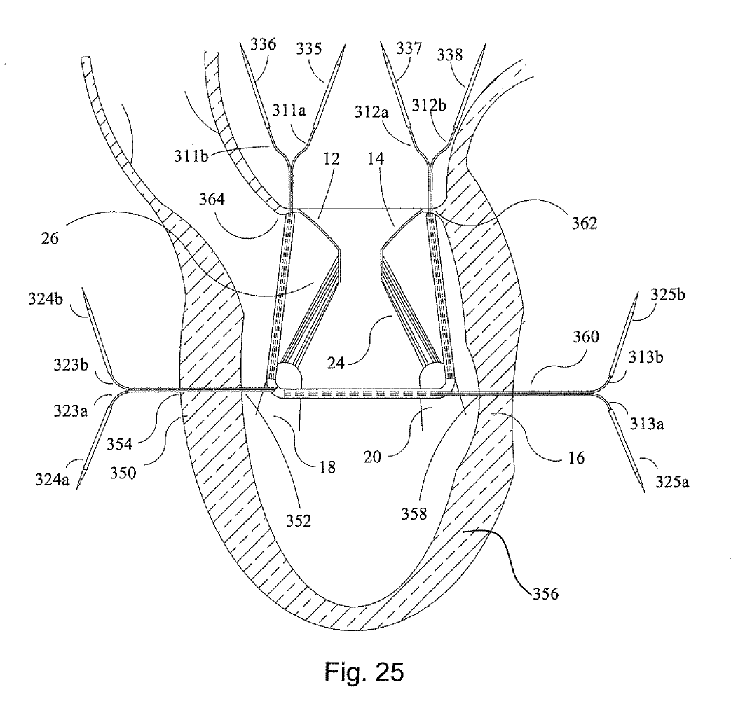

A further embodiment 300 shown in FIGS. 24-25 comprises a ventricular device 310 having a flexible and collapsible housing 320, 321, 322 which in the illustrated embodiment is made from a single length of braided Polyester tube of approximately 2.5 mm outside diameter and approximately 1.25 mm internal diameter.

The flexible, collapsible housing contains three pairs of drawstrings 311, 312, 313 as shown in FIG. 2. The drawstrings may be of size 2 braided Polyester surgical suture with each having a distinct color for easy identification. The drawstring tails or ligatures 311, 312, 313 exit the tubular braid at 14, 15, 16 respectively and are folded and anchored at points 317, 318, 319 at the folds respectively. Drawstring tails 311a, 311b, 312a, 312b may conveniently be terminated in short straight cardiovascular type surgical needles 335, 336, 337, 338 as shown in FIG. 2.

The polyester tube may be heat set (or folded) into a generally “U” shaped form as shown in FIG. 24. When implanted the bottom lateral limb 322 of the device 310 lies in a substantially straight line between the opposing ventricular walls adjacent to the left and right papillary muscles 106, 105. The drawstring loop 313 passes though the lateral portion with the loop anchored to the polyester tube at 319 near the first end of the lateral portion of the housing 320 that lies against and is sewn to the ventricular wall by means of optional suture 323. Alternatively a separate suitable cardiovascular suture anchored in housing 320 in the proximity of points 317 and 319 may be used to anchor the tube limbs 321, 322 approximately to points 317 or 319.

The device is preferably implanted as shown in FIG. 25. The two tails 323a, 323b of suture 323 has needles 324a, 324b that are passed through the ventricular wall 350 at 352, close to papillary muscle 106 to emerge at 354 as shown (though it could be at the base or below the papillary muscles). Suture needles 325a and 325b associated with drawstrings 313a, 313b are passed through ventricular wall 356 at 358, at about the same height as suture 323 to exit the wall at point 360. Drawstrings 311a, 311b, 312a, 312b are passed through the mitral annulus 362, 364. A surgeon may choose to run the sutures 313a, 313b and 323 through to papillary muscle.

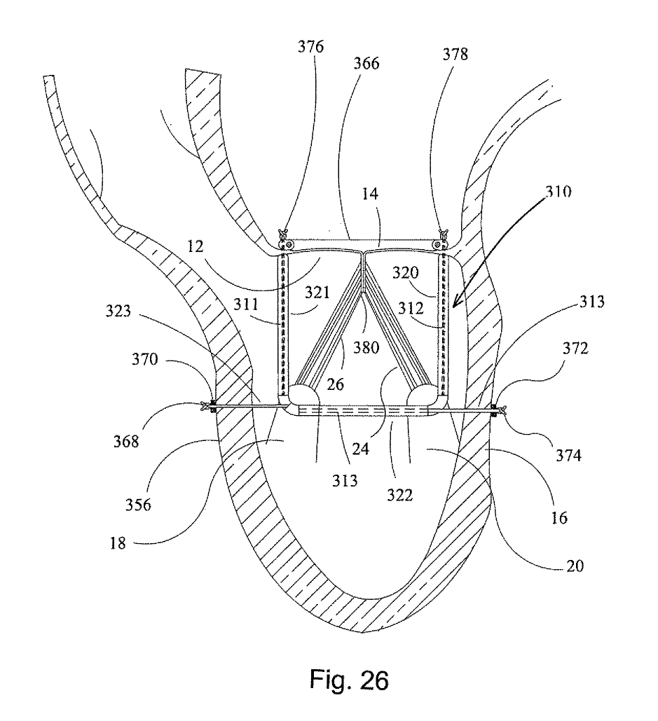

In the embodiment illustrated herein, a rigid mitral annuloplasty ring or band 366 may be implanted at the same time as the device as the annuloplasty ring acts as a load bearing strut to distribute axial downwards pull exerted by drawstrings pairs 312, 313, and prevents unwanted dilation or distortion of the mitral annulus. Alternatively, other structures such as buttons, pledgets, ring segments (rigid or flexible) or a flexible or semi-rigid annuloplasty ring may be used if the annulus is healthy and not distorted, provided they can effectively distribute the axial loads without undue distortion of the mitral annulus. “Undue” means distortion interfering with the long term integrity of the mitral valve. FIG. 26 shows the final implantation of a rigid annuloplasty ring 366 in conjunction with the device 310. However, prior to finalizing the implantation step suture 323 is tied off with knot 368 against pledget 370 and cut, drawstring 313 is tightened (an appropriate amount to draw the two papillary muscles laterally towards each so that they are separated substantially a distance present in a healthy heart (i.e., a “normal” distance). Likewise the other two drawstring pairs 311, 312 are tightened to draw the papillary muscles axially towards the mitral annulus until near normal distances between the apexes of the papillary muscles and the mitral annulus are achieved. Following the initial tightening of the drawstrings it is prudent to test of competency of the valve by injecting liquid, such as physiological saline solution, into the left ventricle at near physiological pressure to demonstrate that the valve leakage has been corrected. Finally the three sets of drawstrings are tied off and trimmed to length, with pledget 372 adjacent knot 374.

The normal implantation sutures used to complete the attachment of the annuloplasty ring to the annulus are not shown. Suture 323 and drawstring 313 knots 368, 374 are preferably reinforced with pledgets 370, 372 or other suitable means such as implantable “buttons”. Drawstring 311 and 312 are terminated in knots 376, 378 abutting the annuloplasty ring 366. It may be seen in FIG. 26 that the leaflets now coapt correctly at 380. The operation may be completed in the normal manner, in conjunction with coronary artery bypass grafting, if appropriate. An advantage of this structure is the fit can be customized to restore the ventricle structure of the papillary muscles and the chordae tendineae to their normal position.

In one embodiment not illustrated herein the suture 323 and the suture 313 can be attached to the lateral portion of the braided polyester near the middle of the braided polyester to act as drawstrings.

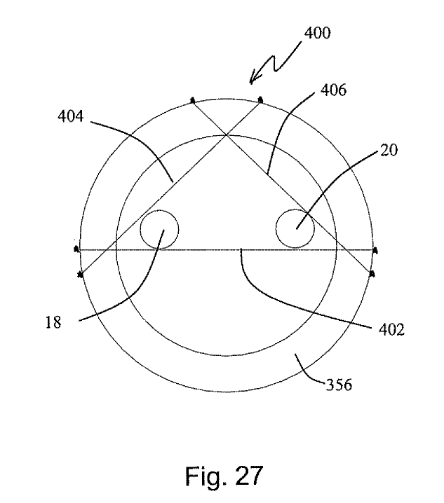

FIG. 27 is a schematic representation of an alternative embodiment of the device described herein. In this embodiment the “base” 400 comprises the lateral limb comprising the suture 313, 323 indicated at 402 as a first lateral limb and second and third lateral limbs 404, 406. The lateral cords 402, 404, 406 form essentially a triangle with two apexes of the triangles formed at or near the point of attachment of the first lateral limb 402 to an outside wall of the ventricle wall and the third apex formed at or near the sidewall of a posterior portion of the ventricle wall intermediate the papillary muscles 105, 106.

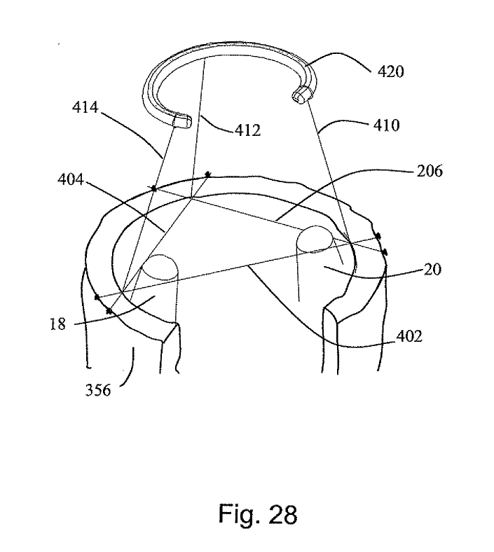

The embodiment 405 may be further modified as illustrated in FIG. 28. In the embodiment illustrated in FIG. 28, the first, second and third lateral limbs formed in the triangle, as discussed above with regard to FIG. 27 have first, second and third axial limbs 410, 412 and 414 extending upward to the mitral annuloplasty ring 420, which would be installed in a mitral annulus. As described above, an annuloplasty ring 420 can be sewn to the mitral annulus and the axial limbs 410, 412 and 414 can be sewn to the annuloplasty ring 420.

The disclosure also encompasses all possible permutations of the claim set, as if the dependent claims were multiple dependent claims of the independent and dependent claims. The disclosure further extends to interpretation of elements of each embodiment into other embodiments.

While the invention has been particularly shown and described with reference to a number of embodiments, it would be understood by those skilled in the art that changes in the form and details may be made to the various embodiments disclosed herein without departing from the spirit and scope of the invention and that the various embodiments disclosed herein are not intended to act as limitations on the scope of the claims. All references cited herein are incorporated in their entirety by reference.

1-67. (canceled)

68. A method for the treatment of mitral regurgitation of a damaged left ventricle of a heart, the left ventricle having at least one of first and second opposing papillary muscles displaced laterally or axially relative to a mitral annulus as compared to normal lateral and axial positions of the opposing papillary muscles relative to a mitral annulus of a healthy heart, the method comprising:
a) halting beating of the heart;
b) attaching opposing ends of a first lateral limb to opposing sides of the ventricle at locations whereby the first and second opposing papillary muscles can be drawn laterally together by shortening the segment of the first lateral limb;
c) shortening the first lateral limb as needed to put the at least one of the first and second opposing papillary muscles into a substantially normal lateral position relative to the mitral annulus and fixing the length of the segment of the first lateral limb;
d) extending a first axial limb attached to the first lateral limb and operatively associating a distal end of the first axial limb with the mitral annulus;
e) extending a second axial limb attached to the first lateral limb and operatively associating a distal end of the second axial limb with the mitral annulus; and
shortening the first and second axial limbs as needed to put the at least one of the first and second opposing papillary muscles into a substantially normal axial position relative to the mitral annulus and fixing the length of the first and second axial limbs.

69. The method of claim 68 further comprising step c) being performed by extending a lateral drawstring operatively associated with the first lateral limb to adjust the length thereof to protrude through the ventricle wall to outside the ventricle and shortening of step c) is performed by tensioning the lateral drawstring to move the ventricle wall inward and the fixing of step c) is performed by securing the lateral drawstring to an outside surface of the ventricle wall.

70. The method of claim 69 further comprising:
g) resuming beating of the heart; and
h) adjusting the length of the first lateral limb by unsecuring the lateral drawstring from the outside surface of the ventricle wall and moving the ventricle wall inward or outward, as desired, and resecuring the drawstring to the outside surface of the ventricle wall.

71. The method of claim 69 further comprising step f) being performed by:
operatively associating a first axial drawstring with the first axial limb to adjust a length thereof and extending the drawstring axially of the first axial limb opposite the mitral annulus through the wall of the ventricle substantially opposite the mitral annulus to outside the ventricle;
operatively associating a second drawstring with the second axial limb to adjust a length thereof and extending the second drawstring axially of the second axial limb opposite the mitral annulus through the wall of the ventricle substantially opposite the mitral annulus to outside the ventricle; and
securing the first and second drawstrings on an outside surface of the ventricle wall with first and second axial limbs at a desired length.

72. The method of 71 further comprising:
g) resuming beating of the heart;
h) adjusting the length of the first lateral limb as needed by unsecuring the drawstring from the outside surface of the ventricle wall and moving the ventricle wall inward or outward, as needed, and resecuring the drawstrings to the outside surface of the heart; and
i) adjusting the length of the first axial limb and the second axial limb as needed by unsecuring the first or second drawstring from the outside surface of the ventricle wall and drawing the first and second drawstrings into or out of the ventricle to lengthen or shorten the first and second axial limbs, respectively, as needed and resecuring the first or second drawstrings on the outside surface of the ventricle wall.

73. The method of claim 68 wherein the limbs comprise surgical sutures.

74. The method of claim 72 wherein, the first and second drawstring each comprise a suture and the operatively associating step comprises folding the suture around a knot in the respective first and second axial limbs formed at a point spaced from the first lateral limb, whereby each suture forms a pair of drawstring ends which extend through the wall of the ventricle substantially opposite the mitral annulus to outside the ventricle.

75. A device for treatment of ischemic mitral regurgitation of a damaged left ventricle having first and second opposing papillary muscles displaced laterally or axially relative to a mitral annulus as compared to a normal lateral and axial positions of opposing papillary muscles relative to a mitral annulus in a healthy heart, the device comprising:
a lateral limb configured to extend between opposing sides of a left ventricle, the lateral limb further being configured to provide for an adjustable length thereof; and
first and second axial limbs extending from spaced points on the lateral limb to form an essentially U-shaped configuration, the first and second axial limbs being configured to extend between the lateral limb and a mitral annulus and the axial limbs further being configured to provide for an adjustable length thereof.

76. The device of claim 75 further comprising the axial and lateral limbs being configured for adjustable length thereof from outside of a beating heart.

77. The device of claim 75 wherein the lateral limb and the first and second axial limbs further comprise a biocompatible, axially compressible tubular housing extending axially of each limb.

78. The device of claim 77 wherein the tubular housing is heat set in the essentially U-shaped configuration.

79. The device of claim 77 further comprising the lateral limb being configured to provide for an adjustable length by comprising at least one drawstring joined near an axial limb to the tubular housing with a free end extending near a midpoint of the lateral limb to provide for the adjustable length.

80. The device of claim 77 further comprising the first and second axial limbs being configured to provide for an adjustable length by each comprising a pair of drawstrings joined to the tubular housing near the lateral limb with the free ends extending axially from distal ends of the axial limbs to provide for the adjustable length.

81. The device of claim 80 further comprising loops formed in the distal ends of the housing of the axial limbs with each free end of the drawstring extending from a different portion of the loop to be spaced from the other.

82. The device of claim 75 further comprising an annuloplasty ring operatively associated with the first and second axial limbs.

83. The device of claim 75 further comprising tabs attached to the axial limbs near the lateral limb, the tabs being configured to wrap around a papillary muscle adjacent thereto.

84. The device of claim 76 further comprising the lateral limb and the first and second axial limbs comprising a biocompatible axially compressible housing, the lateral limb being configured for adjustable length thereof from outside of a beating heart by a folded suture defining a drawstring pair joined to the lateral housing at the suture fold near an axial limb with free ends of the drawstring pair extending from the housing.

85. The device of claim 76 further comprising the first and second axial limbs being configured for adjustable length thereof from outside of a beating heart by each comprising a first folded suture defining a first drawstring pair joined to the axial limb housing at the suture fold near the lateral limb with free ends of the drawstring pair extending axially from a distal end of the axial housing, the first pair of drawstrings each having a axle operatively associated therewith at a point within the axial housing intermediate a proximal end of the housing and the distal end of the housing, the first and second axial limbs further comprising a second folded suture defining a second drawstring pair, the second folded suture being draped over the axle with free ends of the second drawstring pair extending axially from the proximal ends of the axial housing.

86. The device of claim 85 further comprising the lateral limb being configured for adjustable length thereof from outside of a beating heart by comprising a folded suture defining a drawstring pair joined to the lateral limb housing near an axial limb at the suture fold with free ends of the drawstring pair extending axially from the lateral limb proximate an axial limb joined thereto.

87. The device of claim 86 further comprising tabs attached to the axial limb housings near the lateral limb, the tabs being configured to wrap around a papillary muscle adjacent thereto.
